(19) United States
(12) Patent Application Publication
(10) Pub. No.: US 2009/0131687 A1
(43) Pub. Date: May 21, 2009

(54) Method of storing and transporting vinylene carbonate

(76) Inventor: Reinhard Langer, Tonisvorst (DE)

Correspondence Address: Michael A Miller;Lanxess Corporation, Law & Intellectual Property Depatment 111 RIDC, West Drive, Pittsburgh, PA, 15275, US

(21) Appl. No.: 11/920,170

(22) PCT Filed: May 4, 2006
(86) PCT No.: PCT/EP2006/004154

§ 371 Date: Dec. 17, 2008

(30) Foreign Application Priority Data
May 12, 2005 (DE) 10-2005-021-965.9

(51) Int. Cl. C07D 317/34

(52) U.S. Cl. 549/230

(57) ABSTRACT

Method for the storage and transport of vinylene carbonate, characterized in that the vinylene carbonate has a stabilizer content of less than 100 ppm and has a purity of 99.9 to 99.99999% and is present in the solid state of aggregation.

The present invention relates to a method for the storage and the transport of vinylene carbonate (VC).

Vinylene carbonate is an important intermediate for the production of chemicals, pharmaceutical products, crop protection agents and in particular for polymers, coatings and battery electrolytes.

Vinylene carbonate is produced by a known method by eliminating hydrogen chloride from chloroethylene glycol carbonate by means of tertiary amines, in particular triethylamine.

Chloroethylene glycol carbonate (CGC) is obtained by free radical chlorination of ethylene glycol carbonate by means of chlorine or sulphuryl chloride.

The synthesis was first published in 1953 by Newman and Addor (JACS, 1953, page 1263; JACS 1955, page 3789).

Ethylene glycol carbonate (GC) was photochlorinated as such by means of ultraviolet light at 60-70° C., and the resulting CGC was purified by vacuum distillation.

Newman and Addor obtained VC by elimination by means of triethylamine in boiling ether, the mixture having been heated overnight.

The isolation was effected by filtering off the triethylammonium chloride and then carrying out distillation, which gave a crude VC in a yield of 59%, which crude VC had to be purified by further distillation.

JP 2000/026449 describes the elimination in high-boiling solvents (b.p. 170-300° C.). The reaction is explicitly effected with triethylamine in dibutyl carbonate for 20 hours at 50° C. After the ammonium chloride has been filtered off and excess triethylamine distilled off, crude VC is isolated by simple distillation. In order to remove traces of amines, the VC is poured over a silica gel column. Finally, a purifying distillation is carried out. The chlorine content of the VC thus obtained is stated as 29 ppm, whereas comparative samples contain >3000 ppm. The yield is 56%.

DE-A 19 955 944 claims the elimination in GC as a solvent (b.p. 243-244° C.). CGC is initially introduced in GC and reacted in 1.5 hours by addition of triethylamine at 60° C. After distilling off of excess triethylamine at 40° C. and evaporation has been effected via a thin-film evaporator at 100° C., a colourless mixture of VC and GC is obtained in a yield of 73%. No data are given concerning the purity.

After the salts have been filtered off and the solvent and other impurities have been separated off by simple distillation, the reactions of CGC in the liquid phase give a crude vinylene carbonate which is contaminated with residues of chloroacetaldehyde, chloroglycol carbonate, dichloroglycol carbonate and further organic compounds, some of which contain chlorine.

Johnson and Patton describe, in JOC, 1960, page 1042, the reaction of CGC over fixed beds of CaSO₄ catalysts in the gas phase at 250° C. and 50-60 mmHg.

DE-A 1 135 452 describes the HCl elimination of CGC at 300-400° C. The CGC is passed in the gaseous form over an inert support material which is coated with elements of subgroup I, II or VIII of the Periodic Table of the Elements or salts or oxides thereof. Preferably the chlorides of iron, of cobalt and of copper and particularly preferably cadmium chloride, are used. Suitable support materials are pumices and silicates having particle sizes of 4 to 8 mm.

The catalysts are operated as a stationary bed at atmospheric pressure or reduced pressure and at 270 to 450° C., preferably at 300-400° C.

The gas-phase process for the production of vinylene carbonate gives, after a simple distillation, a crude vinylene carbonate which is very similar to the liquid process with regard to impurities.

Regarding the distillative purification effort, the data in the literature are inexact, so that the effort made in the individual case and the losses of yield due to the purification cannot be estimated.

The high purity of the VC is of great industrial importance particularly for the applications of polymerization and as an additive for battery electrolytes.

U.S. Pat. No. 2,873,230 states that, even with an 80-tray column, VC produced by the method of Newman and Addor cannot be sufficiently purified to be copolymerized with vinyl acetate, and insufficient molecular weights are achieved in the homopolymerization. Chlorine-containing impurities are said to be responsible for this.

Huang et al. state, in Chin. J. Polm. Sci. (1990) 8 (3), 197-203, that VC produced by the method of Newman and Addor, after it has been isolated by filtration and the solvent distilled off, is stirred for 1 hour with about 4% of NaBH₄ at 64° C. and only thereafter subjected to a purifying distillation. This procedure must be repeated in order to obtain readily polymerizable material stable to discoloration.

Neither of the two literature references discuss in detail the content of impurities which remain in the pure VC. Losses due to the isolation procedure are likewise not discussed.

GB-A 899 205 describes the purification of VC produced according to Newman and Addor by repeated melt crystallization. In order to obtain polymers having a high molecular weight, it is necessary to use VC having a melting point greater than 21° C., which is obtained by quadruple crystallization. Here too, the purity of the VC is not directly discussed and just as little discussion is devoted to the disposition of the mother liquors. Since the VC has been produced as described in JACS, 75, 1263 (1953), distilled VC was used in the crystallization.

Zief and Ruch describe, in Journal of Chemical Education (1963, Vol. 40, pages 351-2), the purification of VC by zone melting. A monomer having a melting point of 22° C. and a chlorine content of 1-1.8% achieves a chlorine content of 500 ppm after zone melting once, and the VC achieved a chlorine content of 50 ppm after a further 3 passes through the zone melting apparatus. The more contaminated the VC, the more zone melt passes the material requires, and a preceding distillation therefore appears expedient to the authors.

JP 2002-322171 describes the combination of distillation and crystallization for the purification of VC. For the crystallization, solvent mixtures comprising an aromatic component and an aliphatic hydrocarbon are claimed. The yield by distillation and crystallization in the examples is 60 and 83%. The purity is above 99.95%. 400 and 25 ppm of ethylene glycol carbonate and chloride contents of 15 ppm remained as impurities in the VC.

In the applications for purification by crystallization, either larger amounts of solvents are employed or the crystallization process has to be repeated several times in order to achieve high purities.

If solvents are employed, the VC must finally be distilled again if it is intended to remove considerable residues of solvent from the VC.

In the case of transport and storage of VC, too, a stabilizer which prevents the decomposition of VC must always be added.

It should be noted that the methods of analysis for purity determination are not described in detail in the literature, so that the purity data are not unambiguous.

It is an object of the invention to provide a method for the storage and for the transport of vinylene carbonate.

Surprisingly, it was found that VC can advantageously be stored and transported without decomposition if it is present in the solid form in high purity.

The invention relates to a method for the storage and the transport of vinylene carbonate, in which the VC has a stabilizer content of less than 100 ppm, preferably less than 10 ppm, and has a purity of 99.9 to 99.99999%, preferably 99.99 to 99.9999%, and is present in the solid state of aggregation.

This highly pure VC is advantageously obtained by
a) bringing the VC into contact at a temperature in the range of 25 to 180° C. with an organic compound having at least one amidic nitrogen-hydrogen bond,
b) optionally filtering off any precipitated solid,
c) distilling the remaining solution over a column and
   d) obtaining the purified VC from the distillate by crystallization.

In the context of the invention, organic compounds having amidic nitrogen-hydrogen bonds are all aliphatic and aromatic carboxamides which have one or more of the following functional groups of the following formula (I)

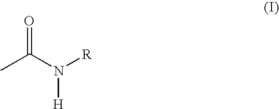

in which R═H, C₁-C₁₀-alkyl or cycloalkyl, C₆-C₁₀-aryl,
or ureas of the following formula (II)

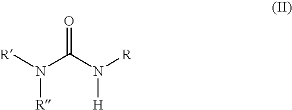

in which R, R′ and R″ are identical or different and are H, C₁-C₁₀-alkyl or cycloalkyl, C₆-C₁₀-aryl, ureas preferably being used.

Organic compounds having amidic nitrogen-hydrogen bonds from the group consisting of formamide, methylformamide, acetamide, methylacetamide, ethylacetamide, phenylacetamide, adipamide, benzamide, phthalamide, propionamide, dimethylurea, diethylurea, diphenylurea, and urea are preferably used. Dimethylurea, diethylurea and diphenylurea are particularly preferably used. Urea is very particularly preferred.

The thermal treatment in step a) is effected with stirring at temperatures between 25 and 180° C., preferably between 60 and 160° C., particularly preferably between 90 and 140° C.

Relative to the vinylene carbonate, 0.1-30% by weight, preferably 1-10% by weight, particularly preferably 2-6% by weight, of purification substance are added.

The addition of the organic compound having at least one amidic natural bond can be effected in the presence of or without addition of solvents. For example, dimethylacetamide, N-methylpyrrolidone (NMP), dimethylformamide and DMSO may be mentioned as solvents.

After the thermal treatment and the filtering off of any precipitated solid, the vinylene carbonate is distilled off from the residue in step c). This can be carried out as a batch distillation from a container via a column having at least 10, preferably at least 20, particularly preferably at least 30, trays.

Suitable column internals are all possibilities known to the person skilled in the art, for example bubble trays, sieve trays and furthermore random packings, such as, for example, Raschig rings, pull rings, Berl saddles and also cross-channel structures, such as, for example, structured packings from Sulzer and Montz.

The low-boiler-rich runnings of the purifying distillation which are obtained by the method according to the invention are advantageously collected, and distilled separately for low-loss discharge of these low boilers, the VC recovered in this manner being recycled for thermal treatment in step a) and/or purifying distillation in step b).

The VC thus obtained is subjected to a melt crystallization in step a) of the method according to the invention, with a content between 99.0 and 99.99, preferably between 99.5 and 99.9%.

The melt crystallization can be carried out in industrial plants, as known to the person skilled in the art; tube-bundle crystallizers may be mentioned in particular.

After the isolation, the VC is obtained in very high purity with residual chloride contents of <10 ppm.

The mother liquor obtained in the crystallization can be recycled directly into the distillation in step c) and/or thermal treatment in step a).

Stabilizer-free pure VC tends to polymerize and is therefore marketed with stabilizers such as BHT.

The purer the monomer, the more sensitive it is to unintended polymer formation.

Surprisingly, it was found that such highly pure VC in the solid state can be stored unchanged over a long time without a stabilizer.

The storage and the transport of highly pure, stabilizer-free VC therefore takes place at a temperature of between −200° C. and +20° C., preferably between 0 and 15° C.

### EXAMPLE

### Production of Solid VC

### Highly Pure

The distillation apparatus consisted of an oil-heated 15 1 pot having a plane-ground joint and an anchor stirrer, column, reflux splitter, condenser and an apparatus for establishing a constant vacuum. A cold trap cooled to −78° C. was present before the vacuum pump. The pot having a plane-ground joint, column, reflux splitter and condenser were made of glass, and the anchor stirrer of Teflon.

The column had a 1500 mm long Sulzer DX structured packing comprising Hastelloy C having a diameter of 50 mm. Structured packings of this type have separation efficiencies of between 15 and 30 trays per metre.

The apparatus was always blanketed with nitrogen before and after loading and before operation.

Crude VC freed substantially only from polymeric impurities by a preliminary distillation without a column was used as starting material.

This crude VC was about 97% pure and had a content of organic and inorganic chlorine of about 0.5% to 1%.

The gas chromatographic analysis was effected by means of an HP 6890. Separation was effected via a 50 metre long CP-Sil 8 CB having an ID of 0.53 mm and an FD of 1.0 μm.

The carrier gas was nitrogen with an admission pressure of 5 psi. The injector was operated with a flow of 138 ml/min and a split of 30/1. 1 μl of pure VC was injected.

The injector temperature was 220° C. and the detector temperature 320° C. The temperature program started at 50° C., with a heating rate of 5° C./min to 250° C. Evaluation was effected by the standard % method.

### Example 1

### Treatment with Urea

200 g of urea were added to 12 060 g of crude VC and the mixture was stirred under nitrogen for 2 hours at 140° C. After cooling to about 30-40° C., 235 g of solid were filtered off, 11 743 g of liquid were transferred into the distillation apparatus described above and 1000 g of NMP were added.

The mixture was refluxed at a pressure of about 35 mbar and then first runnings were distilled off with a reflux ratio of 30:1.

Within 2.5 hours, about 160 g of distillate which, according to GC analysis, comprised 96% of VC were thus obtained. In the following 3.5 hours, about 400 g of a distillate which comprised 97.5% of VC were obtained, followed by about 470 g of distillate having a VC content of 99.4%, which distilled over in 2.5 hours.

The main run was then taken off with a reflux ratio of 5:1. About 9600 g of a 99.9% pure VC which had a chlorine content below 50 ppm distilled in 26 hours.

About 1100 g of bottom product having a VC content of less than 0.5% remained behind.

The cold trap was virtually empty.

The mass balance was virtually quantitative, and 93% of VC was recovered.

84% of the vinylene carbonate were obtained in the main fraction.

### Example 2

### Crystallization in a Static Crystallizer

The crystallizer consisted of a 400 mm long thermostatted glass tube having an internal diameter of 30 mm. A perforated disc for fixing the crystals and a discharge valve for the mother liquor were mounted at the lower end. An argon-flushed interchangeable receiver was present under the shut-off valve. At the upper end, it was possible to replace the gas phase via flushing with argon, and furthermore a coolable plastic finger extended into the interior, by means of which the crystallization could be initiated in a controlled manner.

302 g of the distillate from Example 1 were introduced into the crystallizer and the gas phase was displaced by flushing with argon. The vinylene carbonate was cooled down by means of cold oil circulation at 19° C. Thereafter, the crystallization was started by cooling the cold finger and was allowed to run for 4 hours.

Rapid propagation of a crystal front from the cold finger over the entire heat exchanger surface was observed. The crystals then grew inwards in a strikingly compact manner.

After 4 hours, by opening the valve at the lower end, first the liquid under the perforated screen in the uncooled part of the tube and then the mother liquor are discharged and are collected separately. Thereafter, the receiver was changed again and the oil circulation was heated to 22° C. with the valve open in the course of 1 h with a linear ramp and was kept at 22° C. for a further hour, the crystals being purified by sweating. Finally, the main fraction was melted in a further receiver at 30° C.

The starting material was about 99.9% pure and had a chlorine content of <50 ppm and a water content of about 100 ppm.

24 g of first runnings were collected, with a VC content of 99.87%, a chlorine content of about 110 ppm and a water content of about 230 ppm.

56 g of mother liquor were obtained, with a VC content of 99.8%, a chlorine content of 160 ppm and a water content of 330 ppm.

The sweat weighed 35 g and had a VC content of 99.9%, 70 ppm of chlorine and 110 ppm of water.

The product melt weighed 187 g and had a VC content of 99.99%, was at the limit of detection with 3 ppm of chlorine and contained 10 ppm of water.

Mother liquor, first runnings and sweat can be recycled directly into the urea treatment or the distillation and are therefore not lost.

### Example 3

### Storage of VC

Samples of vinylidene carbonate purified according to Example 2 were stored as a liquid at 20° C. and in crystalline form at 5° C. without a stabilizer and with exclusion of light.

### a) Comparative Experiment:

The stabilizer-free samples of vinylene carbonate show slight to strong yellow colorations and slight turbidity phenomena after 70 days. The content, measured by ISTD, decreased to 96%. Without a stabilizer, highly pure vinylene carbonate in the liquid state is not stable during storage.

### b) According to the Invention:

The samples stored at 5° C. in the solid state were all colourless and clear after melting even after 365 days. The analyses showed no changes in the quality. Stabilizer-free vinylene carbonate is stable during storage in the solid state.

1. Method for the storage and transport of vinylene carbonate, characterized in that the vinylene carbonate has a stabilizer content of less than 100 ppm and has a purity of 99.9 to 99.99999% and is present in the solid state of aggregation.

2. Method according to claim 1, characterized in that the vinylene carbonate is stored or transported at a temperature between −200° C. and +20° C.
